(19) United States
(12) Patent Application Publication
(10) Pub. No.: US 2017/0198054 A1
(43) Pub. Date: Jul. 13, 2017

(54) METHODS FOR TREATING CANCER WITH ANTI BIP OR ANTI MICA ANTIBODIES

(71) Applicant: Dana-Farber Cancer Institute, Boston, MA (US)

(72) Inventors: Christopher HARVEY, Boston, MA (US); Kai WUCHERPFENNIG, Brookline, MA (US); Jason PYRDOL, Brockton, MA (US); Glenn DRANOFF, Lexington, MA (US)

(21) Appl. No.: 15/312,935

(22) PCT Filed: May 21, 2015
(86) PCT No.: PCT/US2015/031951

§ 371 Date: Nov. 21, 2016

Related U.S. Application Data
(60) Provisional application No. 62/001,571, filed on May 21, 2014.

(51) Int. Cl. C07K 16/30; A61K 38/17; C07K 16/28; C07K 16/18

(52) U.S. Cl. CPC C07K 16/30; C07K 16/18; A61K 38/1709; C07K 16/2833; C07K 2317/31; C07K 2317/21

(57) ABSTRACT

The present invention provides method of compositions for treating cancer by inhibiting MIC shedding.

### RELATED APPLICATIONS

This application claims priority to, and the benefit of U.S. provisional Application No. 62/001,571 filed May 21, 2014, the contents of which are incorporated herein by reference in their entireties.

### FIELD OF THE INVENTION

This invention relates generally methods of treating cancer by inhibition of shedding of NKG2D ligands such MHC class I chain related protein A and B.

### BACKGROUND OF THE INVENTION

MHC class I chain related protein A and B are NKG2D ligand shed from tumor cells, i.e., released from the cell surface into the surrounding medium, and sera from a subset of cancer patients contains elevated levels of the soluble form (sMICA). MIC (the term “MIC” referring to MICA and MICB) shedding is accomplished in part through interactions with the protein disulfide isomerase ERp5, which cleaves a disulfide bond in the MIC a3 domain, rendering it susceptible to proteolysis by ADAM-10/17 and MMP14. Methods of treating cancer by administering anti-MIC antibodies or antigen-binding peptide fragments have been described.

Binding immunoglobulin protein (BiP) also known as 78 kDa glucose-regulated protein (GRP-78) or heat shock 70 kDa protein 5 (HSPAS) is a protein that in humans is encoded by the HSPAS gene.

BiP is a HSP70 molecular chaperone located in the lumen of the endoplasmic reticulum (ER) that binds newly synthesized proteins as they are translocated into the ER, and maintains them in a state competent for subsequent folding and oligomerization. BiP is also an essential component of the translocation machinery, as well as playing a role in retrograde transport across the ER membrane of aberrant proteins destined for degradation by the proteasome. BiP is an abundant protein under all growth conditions, but its synthesis is markedly induced under conditions that lead to the accumulation of unfolded polypeptides in the ER.

### SUMMARY OF THE INVENTION

The invention is based upon the discovery of that BiP plays an important role in the shedding of MIC proteins from the surface of cancer cells and thus contributes to immunosuppression in cancer. BiP causes partial unfolding of the MIC α3 domain, rendering the α3 domain sensitive to downstream contributors of shedding (ERp5 and proteases).

In various aspects the invention provides methods of treating or alleviating a symptom of cancer by administering to a subject an effective amount of a BiP modulating composition. The BiP modulating composition is for example a BiP antibody. In some embodiments the method further includes administering an antibody specific for a charperone protein expressed on the surface of a tumor cell. The BiP modulator composition reduces the level of soluble MIC, e.g. MICA or MICB in the subject. By soluble the level of soluble MICA or MICB is meant the level in the serum.

In another aspect the invention provides a method of treating or alleviating a symptom by administering to a subject a BiP peptide in an amount sufficient to induce an anti-BiP immune response. The BiP peptide is conjugated to a carrier protein such as for example tetnus toxin or diphtheria toxin.

In a further aspect, the invention provides method of treating or alleviating a symptom by administering to a subject a BiP protein or fragment thereof In some embodiments the subject has been administered a MIC vaccine. In other aspects the subject is also administered a MIC antibody such as CM24002.

In one aspect the invention provides method of treating or alleviating a symptom of cancer by administering to a subject an effective amount of a bispecific antibody that specifically binds to MIC and BiP.

The invention also includes a method of producing an immunogen by contacting a tumor specific antigen with a BiP polypeptide in an amount sufficient to induce partial unfolding of the antigen.

The invention also provides an immunogen where the immunogen is a cell co-expressing BiP and an antigen. The antigen is an NKG2D ligand such as MIC.

Other features and advantages of the invention will be apparent from and are encompassed by the following detailed description and claims.

### BRIEF DESCRIPTION OF THE DRAWINGS

FIG. 1 shows a model of the effect of BiP/GRP78 (HSP70 family member) on the ER stress responses in a cell. Specifically, BiP/GRP78 acts as a major ER chaperone with anti-apoptotic properties (sequesters caspases 7 and 12). In addition, BiP/GRP78 controls the activation of the transmembrane ER stress sensors (IRE1, PERK, and ATF6) through a binding-release mechanism. Accumulation of unfolded proteins reduces the amount of BiP available for inhibition of the ER stress sensors.

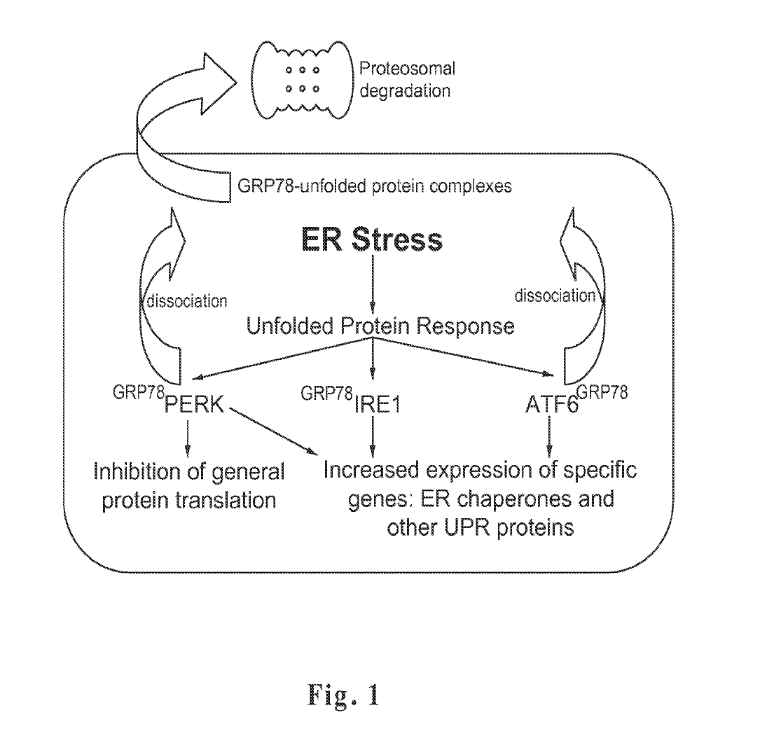

FIG. 2 shows a chart of the role of GRP78 in resistance against therapeutic agents in different cancer types.

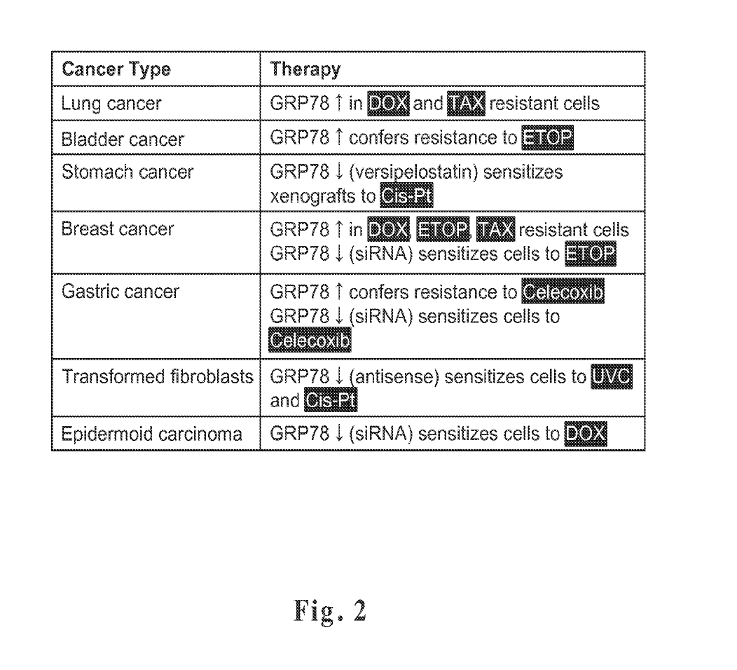

FIG. 3 shows ERp5 and BiP surface staining on U937 and RPMI-8226 cells. Specifically, Panel A shows U937 acute myeloid leukemia cells that were stained for ERp5 and BiP surface expression under basal conditions. Staining was preformed with alexa-647 labeled antibodies and analyzed on a BD FACS Aria. Labeled from top to bottom, U937_BiP corresponds to the top curve, U937_ERp5 corresponds to the second curve from the top, U937_isotype alone corresponds to the second curve from the bottom, and U937_untreated corresponds to the bottom curve. Panel B shows RPMI-8226 multiple myeloma cells that were stained for ERp5 and BiP surface expression under basal conditions. Again, staining was performed with alexa-647 labeled antibodies and analyzed on a BD FACS Aria. Labeled from top to bottom, RPMI-8226_BiP corresponds to the top curve, RPMI-8226_ERp5 corresponds to the second curve from the top, RPMI-8226_isotype alone corresponds to the second curve from the bottom, and RPMI-8226_untreated corresponds to the bottom curve.

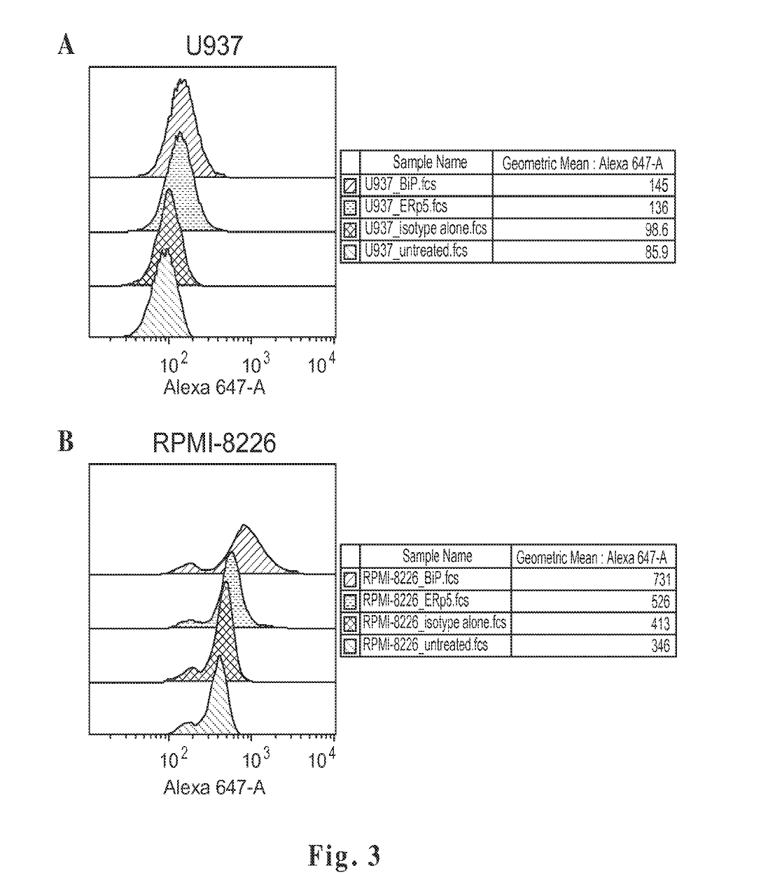

FIG. 4 shows surface stabilization and reduction of MICA shedding in BiP treated RPMI-8226 cells. RPMI-8226 cells were treated with anti-BiP polyclonal antibody at 10 ug/ml for 48 hr. After treatment, soluble MICA in culture supernatant was determined by sandwich ELISA (Panel A, bar chart) and surface staining of MICA was determined by flow cytometry (Panel B). For Panel B, labeled from top to bottom, RPMI-8226 cells_BIP-BL stained corresponds to the top curve, RPMI-8226 cell MICA biolegend-NT corresponds to the second curve from the top, and RPMI-8226 cells unstained corresponds to the bottom curve.

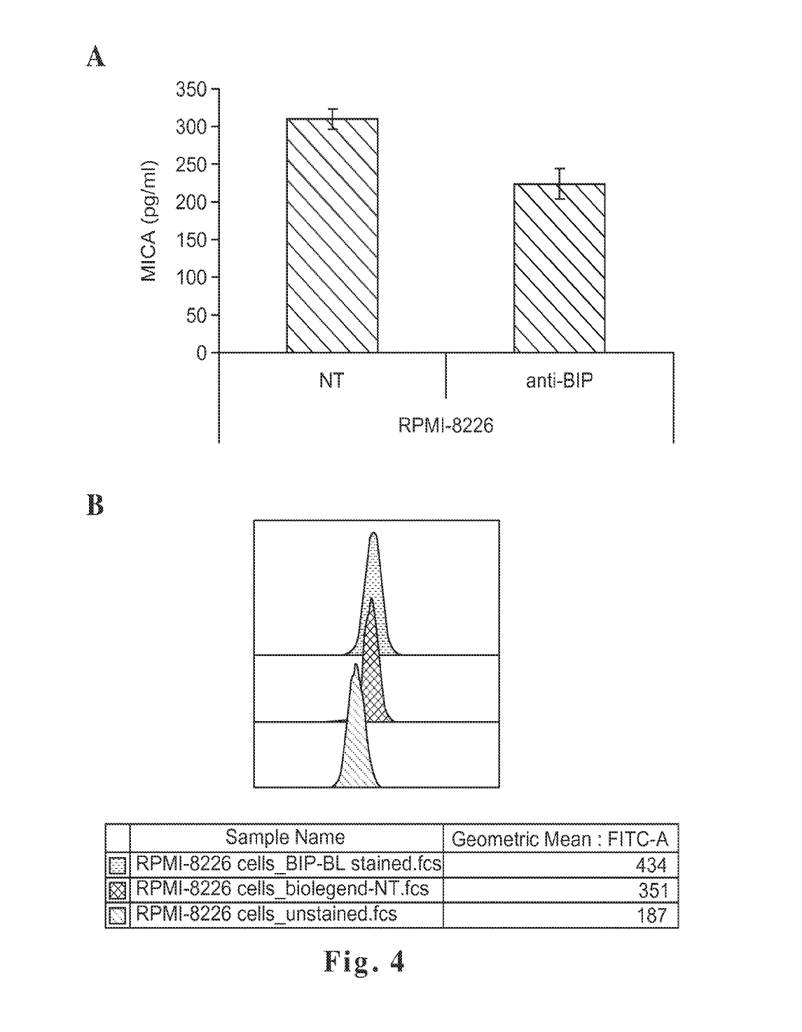

FIG. 5 shows surface stabilization in BiP treated RPMI-8226 cells. RPMI-8226 cells were treated with a titration of anti-BiP polyclonal or monoclonal antibodies. After treatment, surface staining of MICA was determined by flow cytometry. Specifically, Panel A shows (labeled from top to bottom) RPMI-8226_polyclonal 3183S 50 ug-BL stained (top curve), RPMI-8226_polyclonal 3183S 10 ug-BL stained (second curve from the top), RPMI-8226_polyclonal 3183S 1 ug-BL stained (third curve from the top), RPMI-8226_isotype treatment-BL stained (second curve from the bottom), and RPMI-8226_unstained (bottom curve). Panel B shows (labeled from top to bottom) RPMI-8226_monoclonal 76-E6 50 ug-BL stained (top curve), RPMI-8226_monoclonal 76-E6 10 ug-BL stained (second curve from the top), RPMI-8226_monoclonal 76-E6 1 ug-BL stained (third curve from the top), RPMI-8226_isotype treatment-BL stained (second curve from the bottom), and RPMI-8226_unstained (bottom curve). Panel C shows (labeled from top to bottom) RPMI-8226_monoclonal EPR4040 50 ug-BL stained (top curve), RPMI-8226_monoclonal EPR4040 10 ug-BL stained (second curve from the top), RPMI-8226_monoclonal EPR4040 1 ug-BL stained (third curve from the top), RPMI-8226_isotype treatment-BL stained (second curve from the bottom), and RPMI-8226_unstained (bottom curve).

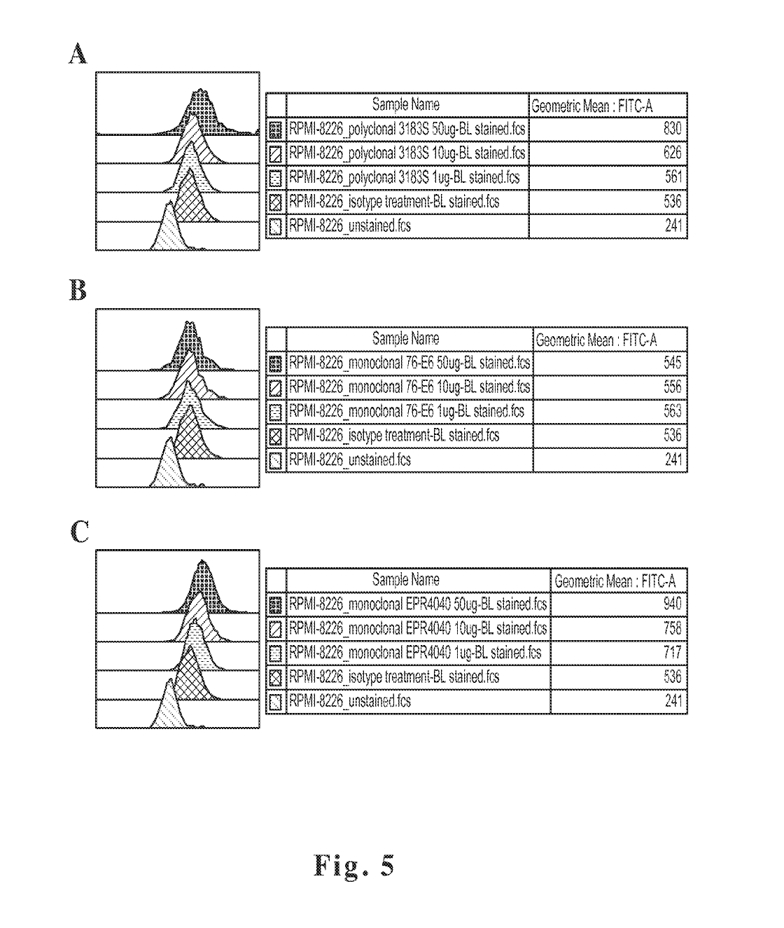

FIG. 6 shows a MICA shedding bar chart. Specifically, RPMI-8226 cells were treated with a titration of anti-BiP polyclonal (3183S) or monoclonal (Mono1=76-E6 and Mono2=EPR4040) antibodies, as characterized in the FIG. 11 description above. After treatment, soluble MICA in culture supernatant was determined by sandwich ELISA. As shown, MICA shedding was reduced in BiP treated RPMI-8226 cells.

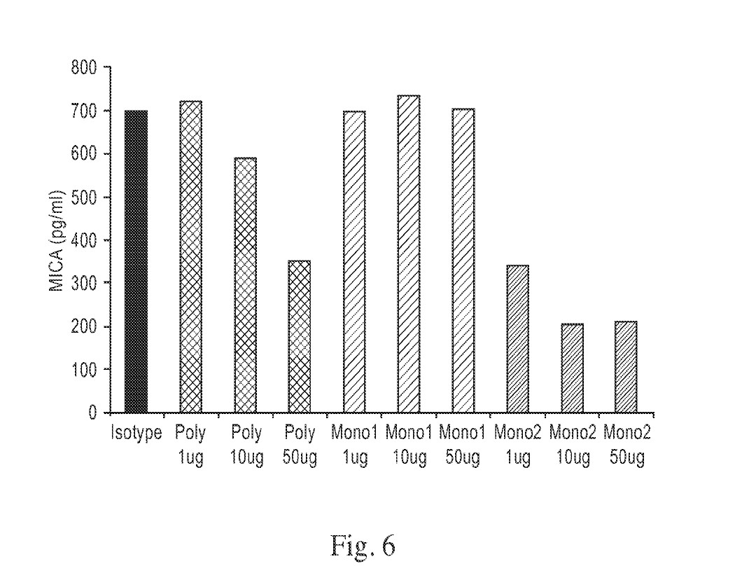

FIG. 7 shows a bar chart of the binding of an isotype control antibody or MIC-specific CM24002 Ab2 antibody to MICA in the presence or absence of BIP. Specifically, biotinylated MICA*002, *008, or *009 was incubated with or without BiP. MICA of the various treatments was captured to wells of a streptavidin coated ELISA plate. Isotype control or CM24002 Ab2 was then incubated with the captured MICAs at 10 ug/ml. Binding of antibodies to MICA was determined with anti-human Europium. As shown, CM24002 Ab2 showed higher binding to MICA in the presence of BIP.

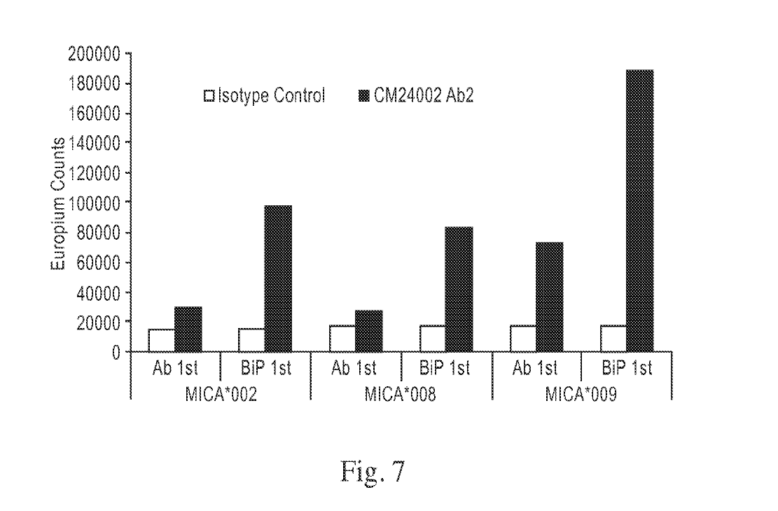

FIG. 8 shows a plot of the binding of an isotype control antibody or CM24002 Ab2 antibody to MICA*002 with different ratios of BiP. Specifically, biotinylated MICA*002 was incubated with the indicated molar ratios of BiP to MICA for 1 hr at 37 C. After incubation, MICA of the various treatments was captured to wells of a streptavidin coated ELISA plate. Isotype control or CM24002 Ab2 was then incubated with the captured MICAs at 10 ug/ml. Binding of antibodies to MICA was determined with anti-human Europium. As shown, treatment of MICA*002 with BiP enhanced binding of CM24002 Ab2.

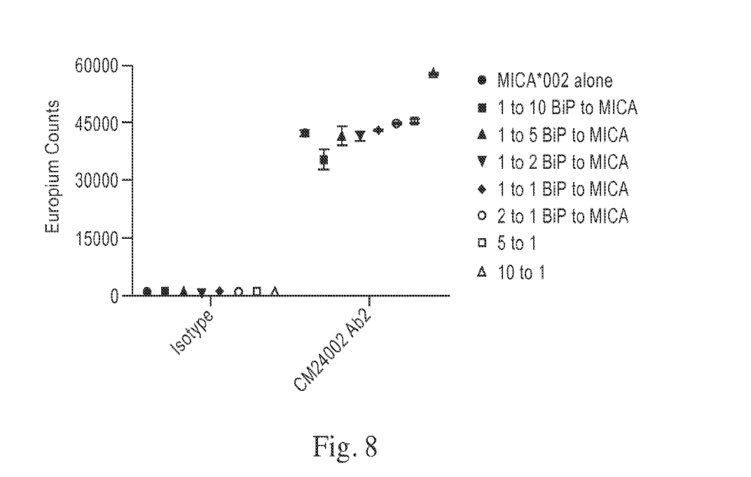

FIG. 9 shows a bar chart (labeled in pairs from left to right) of the binding of a control antibody (TTCF), CM24002 Ab2 antibody, CM33322 Ab28 antibody, CM33322 Ab29 antibody, or CM33322 Ab22 antibody to MICA*002 (left bar) or MICA*002+BiP (right bar). Specifically, biotinylated MICA*002 was incubated at a 1:10 molar ratio of BiP to MICA for 16 hr at 37 C. After incubation, treated or untreated MICA was captured to wells of a streptavidin coated ELISA plate. The indicated antibodies were then incubated with the captured MICAs at 10 ug/ml. Binding of antibodies to MICA was determined with anti-human Europium. As shown, treatment of MICA*002 with BiP enhanced binding of CM24002 Ab2 and CM33322 Ab22.

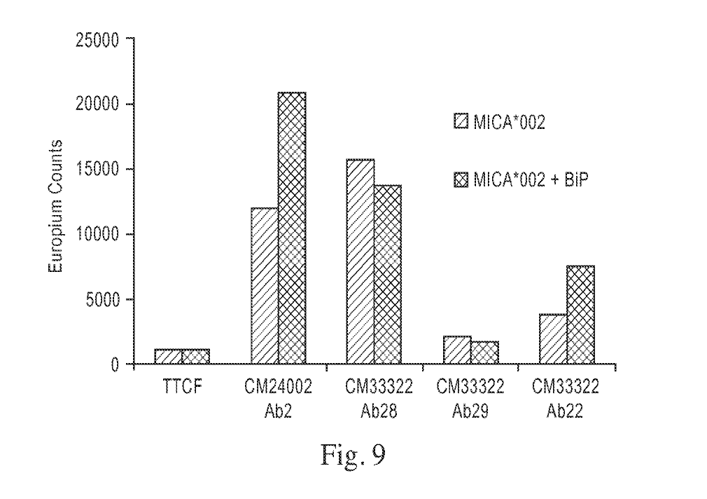

### DETAILED DESCRIPTION

The present invention is based in part upon the surprising discovery that Binding immunoglobulin protein (BiP) enhances the binding of anti-MHC class I chain related protein A (MICA) antibodies to MICA. More specifically, the invention is based upon the further discovery that antibodies to BiP stabilized and reduced the shedding of MICA. Accordingly, the invention provides methods of treating diseases and disorders associated with MIC A/B shedding. In addition the invention provides BiP modulating compositions such as BiP antibodies and compositions and methods for eliciting an immune response against BiP.

### BiP-Modulating Compositions

Provided herein are BiP-modulating compositions. As used herein, the term “BiP modulating composition” refers to any composition that specifically binds to BiP and decrease in the level of soluble NKG2D ligands such as MIC relative to the levels of soluble NKG2D ligands such as MIC in a biological sample that has not been exposed to the BiP modulating compound. MIC is an MHC class I related polypeptide expressed on the surface of many different types of cancer cells. Preferred BiP modulators are inhibitors of one or more activities of NKG2D ligands, such as, specifically inhibit the shedding of NKG2D ligand proteins by cancer cells. Most preferred BiP modulators are inhibitors of one or more activities of MIC, such as, specifically inhibit the shedding of MIC proteins by cancer cells.

Double stranded DNA breaks trigger high-level expression of MIC in a broad range of human cancers, including melanoma, lung, breast, kidney, ovarian, prostate, gastric, pancreatic and colon carcinomas as well as plasma cell cancer, leukemias and lymphomas. MIC is typically localized on the cell surface. MIC is also shed by tumor cells, i e, released from the cell surface into the surrounding medium, and sera from a subset of cancer patients typically contain elevated levels of the soluble form (sMICA/B) Shed MICA/B is thought to impair host defense by inducing the internalization of NKD2G molecules on lymphocytes (CD8 T-cells and NK cells). Shedding generally involves the cleavage and release of a soluble ectodomain from membrane bound proteins, MIC shedding is promoted by a protein disulfide isomerase (PDI), ERp5 PDIs are normally localized in the endoplasmic reticulum but can be transported to the surface of cancer cells where they can disrupt disulphide bonds. The surface localization of ERp5 in cancer cells renders the α3 domain of MIC susceptible to proteolysis, the release of soluble ligand in turn provokes the down regulation of NKG2D.

A BiP-modulating composition can include anti-BiP antibody. As used herein, useful antibodies can include monoclonal and polyclonal antibodies, single chain antibodies, chimenc antibodies, bifunctional/bispecific antibodies, humanized antibodies, human antibodies, and complementary determining region (CDR)-grafted antibodies, that are specific for the target protein or fragments thereof, and also include antibody fragments, including Fab, Fab, F(ab′)2, scFv, Fv, camelbodies, or microantibodies.

Monoclonal antibodies are homogeneous antibodies of identical antigenic specificity produced by a single clone of antibody producing cells. Polyclonal antibodies generally can recognize different epitopes on the same antigen and that are produced by more than one clone of antibody producing cells Each monoclonal antibody is directed against a single determinant on the antigen In addition to their specificity, the monoclonal antibodies are advantageous in that they may be synthesized uncontaminated by other antibodies. The modifier, monoclonal, indicates the character of the antibody as being obtained from a substantially homogeneous population of antibodies, and is not to be construed as requiring production of the antibody by any particular method For example, the monoclonal antibodies may be made by the hybridoma method first described by Kohler et al, Nature, 256 495 (1975), or may be made by recombinant DNA methods (see, e g, U.S. Pat. No. 4,816,567). The monoclonal antibodies may also be isolated from phage antibody libraries using the techniques described in Clackson et al, Nature, 352 624-628 (1991) and Marks et al, J Mol Biol, 222 581-597 (1991), for example.

The monoclonal antibodies herein can include chimeric antibodies, i e, antibodies that typically have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No.

4,816,567, and Morrison et al, Proc Natl Acad Sci USA, 81 6851-6855 (1984)) Chimeric antibodies of interest include humanized antibodies comprising variable domain antigen-binding sequences derived from a non-human primate (e.g. apes, Old World monkeys, New World monkeys,) and human constant region sequences.

Antibody fragments generally include a portion of an intact antibody in some embodiments, the portion of an intact antibody can be the antigen-binding or variable region of the corresponding intact antibody Examples of antibody fragments include Fab, Fab′, F(ab′)2, and Fv fragments, diabodies, linear antibodies (Zapata et al, Protein Eng 8(10) 1057-1062 [1995]), single chain antibody molecules, and multispecific antibodies formed from antibody fragment(s).

An intact antibody is one that comprises an antigen-binding variable region as well as a light chain constant domain (C_{L}) and heavy chain constant domains, C_{HI}, Cm and C_{H3} The constant domains may be native sequence constant domains (e g human native sequence constant domains) or ammo acid sequence variants thereof. In some embodiments the intact antibody has one or more effector functions.

A wide variety of antibody/immunoglobulin frameworks or scaffolds can be employed so long as the resulting polypeptide includes at least one binding region that is specific for the target protein Such frameworks or scaffolds include the five main idiotypes of human immunoglobulins, or fragments thereof (such as those disclosed elsewhere herein), and include immunoglobulins of other animal species, preferably having humanized aspects Single heavy-chain antibodies such as those identified in camelids are of particular interest in this regard novel frameworks, scaffolds and fragments continue to be discovered and developed by those skilled in the art. One can generate non-immunoglobulin based antibodies using non-immunoglobulin scaffolds onto which CDRs of the anti-BiP antibody can be grafted. Any non-immunoglobulin framework and scaffold know to those in the art may be used, as long as the framework or scaffold includes a binding region specific for the target Examples of non-immunoglobulin frameworks or scaffolds include, but are not limited to, Adnectins (fibronectin) (Compound Therapeutics, Inc, Waltham, Mass.), ankyin (Molecular Partners AG, Zuπch, Switzerland), domain antibodies (Domantis, Ltd (Cambridge, Mass.) and Ablynx NV (Zwijnaarde, Belgium)), lipocahn (Anticalm) (Piers Proteolab AG, Freismg, Germany), small modular Immuno-pharmaceuticals (Trubion Pharmaceuticals Inc, Seattle, Wash.), maxybodies (Avidia, Inc (Mountain View, Calif.)), Protem A (Affibody AG, Sweden) and affilm (gamma-crystalin or ubiquitin) (Sell Proteins GmbH, Halle, Germany).

The term polypeptide as used herein refers to a compound of two or more subunit amino acids, amino acid analogs, or other peptidomimetics, regardless of post-translational modification, e g, phosphorylation or glycosylation The subunits may be linked by peptide bonds or other bonds such as, for example, ester or ether bonds The term “amino acid” refers to natural and/or unnatural or synthetic ammo acids, including D/L optical isomers Full-length proteins, analogs, mutants, and fragments thereof are encompassed by this definition.

The anti-BiP antibody can be a monoclonal antibody, a polyclonal antibody, a chimeric antibody, a human antibody, a humanized antibody, a single-chain antibody, or an Fab fragment. In some embodiments the antibody has a binding affinity less than about 1×10⁵Ka for a polypeptide other than BiP. In some embodiments, the anti-BiP antibody or is a monoclonal antibody which binds to BiP with an affinity of at least 1×10⁸Ka Monoclonal antibodies can be prepared using the method of Kohler et al (1975) Nature 256 495-496, or a modification thereof. Typically, a mouse is immunized with a solution containing an antigen Immunization can be performed by mixing or emulsifying the antigen-containing solution in saline, m some embodiments in an adjuvant such as Freund's complete adjuvant, and injecting the mixture or emulsion parenterally. Any method of immunization known in the art may be used to obtain the monoclonal antibodies After immunization of the animal, the spleen (and optionally, several large lymph nodes) are removed and dissociated into single cells. The spleen cells may be screened by applying a cell suspension to a plate or well coated with the antigen of interest The B cells expressing membrane bound immunoglobulin specific for the antigen bind to the plate and are not rinsed away. Resulting B cells, or all dissociated spleen cells, are then induced to fuse with myeloma cells to from hybridomas, and are cultured in a selective medium. The resulting cells are plated by serial or limiting dilution and are assayed for the production of antibodies that specifically bind the antigen of interest (and that do not bind to unrelated antigens). The selected monoclonal antibody (mAb)-secreting hybridomas are then cultured either in vitro (e g, in tissue culture bottles or hollow fiber reactors), or in vivo (as ascites in mice).

In some embodiments the anti-BiP antibody is a humanized antibody Human antibodies can be produced using techniques known m the art, including phage display libraries (Hoogenboom and Winter, J Mol Biol, 227 381 (1991), Marks et al, J Mol Biol, 222 581 (1991)) The techniques of Cole et al and Boerner et al are also available for the preparation of human monoclonal antibodies (Cole et al, Monoclonal Antibodies and Cancer Therapy, Alan R Liss, p 77 (1985) and Boerner et al, J Immunol, 147(1) 86 95 (1991)).

Humanized antibodies may be engineered by a variety of methods including, for example (1) grafting the non-human complementarity determining regions (CDRs) onto a human framework and constant region (a process referred to in the art as humanizing), or, alternatively, (2) transplanting the entire non-human variable domains, but providing them with a human-like surface by replacement of surface residues (a process referred to in the art as veneering). Humanized antibodies can include both humanized and veneered antibodies. Similarly, human antibodies can be made by introducing human immunoglobulin loci into transgenic animals, e g, mice in which the endogenous immunoglobulin genes have been partially or completely inactivated. Upon challenge, human antibody production is observed, which closely resembles that seen in humans m all respects, including gene rearrangement, assembly, and antibody repertoire. This approach is described, for example, in U.S. Pat. Nos. 5,545,807, 5,545,806, 5,569,825, 5,625,126, 5,633,425, 5,661,016, and m the following scientific publications Marks et al, Bio/Technology 10, 779-783 (1992), Lonberg et al, Nature 368 856-859 (1994),

Morrison, Nature 368, 812-13 (1994), Fishwild et al, Nature Biotechnology 14, 845-51 (1996), Neuberger, Nature Biotechnology 14, 826 (1996), Lonberg and Huszar, Intern Rev Immunol 13 65-93 (1995), Jones et al, Nature 321 522-525 (1986), Momson et al, Proc Natl Acad Sci. U S A. 81 6851-6855 (1984), Momson and Oi, Adv Immunol, 44 65 92 (1988), Verhoeyer et al, Science 239 1534 1536 (1988), Padlan, Molec Immun 28 489-498 (1991), Padlan, Molec Immunol 31(3) 169-217 (1994), and Kettleborough, C A et al, Protein Eng 4(7) 773 83 (1991) each of which is incorporated herein by reference.

In addition to chimeric and humanized antibodies, fully human antibodies can be derived from transgenic mice having human immunoglobulin genes (see, e g, U.S. Pat. Nos. 6,075,181, 6,091,001, and 6,114,598, all of which are incorporated herein by reference), or from phage display libraries of human immunoglobulin genes (see, e g McCafferty et al, Nature, 348 552-554 (1990) Clackson et al, Nature, 352 624 628 (1991), and Marks et al, J Mol Biol, 222 581-597 (1991)). In some embodiments, antibodies may be produced and identified by scFv-phage display libraries Antibody phage display technology is available from commercial sources such as from Morphosys.

As an alternative to the use of hybridomas for expression, antibodies can be produced in a cell line such as a CHO or myeloma cell line, as disclosed in U.S. Pat. Nos. 5,545,403, 5,545,405, and 5,998,144, each incorporated herein by reference. Briefly the cell line is transfected with vectors capable of expressing a light chain and a heavy chain, respectively. By transfecting the two proteins on separate vectors, chimeric antibodies can be produced Immunol 147 8, Banchereau et al (1991) Clm Immunol Spectrum 3 8, and Banchereau et al (1991) Science 251 70, all of which are herein incorporated by reference

A complementarity determining region of an antibody typically includes ammo acid sequences that together define the binding affinity and specificity of the natural Fv region of a native immunoglobulin binding site See, e g, Chothia et al, J Mol Biol 196 901-917 (1987), Kabat et al, U S Dept of Health and Human Services NIH.

Publication No 91 3242 (1991) A constant region of an antibody typically includes the portion of the antibody molecule that confers effector functions, including for example, the portion that binds to the Fc receptor on dendritic cells. In some embodiments, mouse constant regions can be substituted by human constant regions For example, the constant regions of humanized antibodies are derived from human immunoglobulins. The heavy chain constant region can be selected from any of the five isotypes alpha, delta, epsilon, gamma or mu. One method of humanizing antibodies includes aligning the non human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region that disturb the structure of the CDRs until the predicted conformation of the humanized sequence model closely approximates the conformation of the non-human CDRs of the parent non-human antibody Such humanized antibodies may be further derivatized to facilitate uptake and clearance, e g, via Ashwell receptors. See, eg, U.S. Pat. Nos. 5,530,101 and 5,585,089 which are incorporated herein by reference.

Human antibodies can also be produced using transgenic animals that are engineered to contain human immunoglobulin loci For example, WO 98/24893 discloses transgenic animals having a human Ig locus wherein the animals do not produce functional endogenous immunoglobulins due to the inactivation of endogenous heavy and light chain loci WO 91/10741 also discloses transgenic non-primate mammalian hosts capable of mounting an immune response to an immunogen, wherein the antibodies have primate constant and/or variable regions, and wherein the endogenous immunoglobuhn-encoding loci are substituted or inactivated.

Cre/Lox system to modify the immunoglobulin locus in a mammal, such as to replace all or a portion of the constant or variable region to form a modified antibody molecule WO 94/02602 discloses non-human mammalian hosts having inactivated endogenous Ig loci and functional human Ig loci U.S. Pat. No. 5,939,598 discloses methods of making transgenic mice m which the mice lack endogenous heavy chains, and express an exogenous immunoglobulin locus comprising one or more xenogeneic constant regions. Antibodies can also be produced using human engineering techniques as discussed m U.S. Pat. No. 5,766,886, which is incorporated herein by reference.

Using a transgenic animal described above, an immune response can be produced to a selected antigenic molecule, and antibody-producing cells can be removed from the animal and used to produce hybridomas that secrete human monoclonal antibodies Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein.

Fragments of antibodies are suitable for use in the methods provided so long as they retain the desired affinity and specificity of the full-length antibody. Thus, a fragment of an anti-BiP antibody will retain an ability to bind to BiP in the Fv portion and the ability to bind the Fc receptor on dendritic cells in the FC portion. Such fragments are characterized by properties similar to the corresponding full-length anti-BiP antibody, that is, the fragments will specifically bind a human BiP antigen expressed on the surface of a human cell. Also provided are antibodies that are SMIPs or binding domain immunoglobulin fusion proteins specific for target protein. These constructs are single-chain polypeptides comprising antigen binding domains fused to immunoglobulin domains necessary to carry out antibody effector functions See e g, WO03/041600, U S Patent publication 20030133939 and US Patent Publication 20030118592.

Any form of the BiP polypeptide can be used to generate anti-BiP including the full length polypeptide or epitope-bearing fragments thereof. Highly suitable anti-BiP antibodies are those of sufficient affinity and specificity to recognize and bind to BiP and in vivo. As used herein, the term epitope refers to an antigenic determinant of a polypeptide In some embodiments an epitope may comprises 3 or more ammo acids in a spatial conformation which is unique to the epitope. In some embodiments epitopes are linear or conformational epitopes. Generally an epitope consists of at least 4, at least 6, at least 8, at least 10, and at least 12 such amino acids, and more usually, consists of at least 8-10 such ammo acids. Methods of determining the spatial conformation of amino acids are known in the art, and include, for example, x-ray crystallography and 2-dimensional nuclear magnetic resonance.

In some embodiments, the antibodies specifically bind to one or more epitopes in an extracellular domain of BiP. Suitable antibodies can recognize linear or conformational epitopes, or combinations thereof It is to be understood that these peptides may not necessarily precisely map to one epitope, but may also contain an BiP sequence, respectively, that is not immunogenic.

Methods of predicting other potential epitopes to which an antibody can bind are well-known to those of skill in the art and include without limitation, Kyte-Doolittle Analysis (Kyte, J and Dohttle, R F, J Mol Biol (1982) 157 105-132), Hopp and Woods Analysis (Hopp, T P and Woods, K R, Proc Natl Acad Sci USA (1981)78 3824-3828, Hopp, T J and Woods, K R, Mol Immunol (1983) 20 483-489, Hopp, T J, J Immunol Methods (1986) 88 1-18), Jameson- Wolf Analysis (Jameson, B A and Wolf, H, Comput Appl Biosci (1988) 4 181-186), and Emini Analysis (Emim, E A, Schhef, W A, Colonno, R J and Wimmer, E, Virology (1985) 140 13-20).

In some embodiments, potential epitopes are identified by determining theoretical extracellular domains. Analysis algorithms such as TMpred (see K Hofmann & W Stoffel (1993) TMbase—A database of membrane spanning proteins segments Biol Chem Hoppe-Seyler 374,166) or TMHMM (A Krogh, B Larsson, G von Heijne, and E L L Sonnhammer Predicting transmembrane protein topology with a hidden Markov model Application to complete genomes Journal of Molecular Biology, 305(3) 567-580, January 2001) can be used to make such predictions Other algorithms, such as SignalP 3.0 (Bednsten et al, (2004) J Mol Biol 2004 Jul. 16, 340(4) 783-95) can be used to predict the presence of signal peptides and to predict where those peptides would be cleaved from the full-length protein. The portions of the proteins on the outside of the cell can serve as targets for antibody interaction.

Specifically binding antibodies are can be antibodies that 1) exhibit a threshold level of binding activity, and/or 2) do not significantly cross-react with known related polypeptide molecules. The binding affinity of an antibody can be readily determined by one of ordinary skill in the art, for example, by Scatchard analysis (Scatchard, Ann NY Acad Sci 51 660-672, 1949). In some embodiments the antibodies can bind to their target epitopes or mimetic decoys at least 1 5-fold, 2-fold, 5-fold 10-fold, 100-fold, 10³-fold, 10⁴-fold, 10⁵-fold, 10⁶-fold or greater for the target cancer-associated polypeptide than to other proteins predicted to have some homology to BiP.

In some embodiments the antibodies bind with high affinity of 10⁴M or less, 10⁷M or less, 10⁹M or less or with subnanomolar affinity In some embodiments the binding affinity of the antibodies for BiP is at least 1×10⁶ Ka In some embodiments the binding affinity of the antibodies for BiP is at least 5×10⁶ Ka, at least 1×10⁷ Ka, at least 2×10⁷ Ka, at least 1×10⁸ Ka, or greater. Antibodies may also be described or specified in terms of their binding affinity to a BiP polypeptide. In some embodiments binding affinities include those with a K_{d} less than 5×10² M, 10² M, 5×10³ M, 10³ M, 5×10⁴ M, 10⁴ M, 5×10⁵ M, 10⁵ M, 5×10⁶ M, 10⁶ M, 5×10⁷ M, 10⁷ M, 5×10^{s} M, 10⁸ M, 5×10⁹ M, 10⁹ M, 5×10¹⁰ M, 10¹⁰ M, 5×10^{π} M, 10^{u} M, 5×10¹² M, 10¹² M, 5×10¹³ M, 10¹³ M, 5×10¹⁴ M, 10¹⁴ M, 5×10¹⁵ M, or 10^{′5} M₃ or less.

In some embodiments, the antibodies do not bind to known related polypeptide molecules, for example, they bind BiP polypeptide o but not known related polypeptides using a standard immunoblot analysis (Ausubel et al, Cunent Protocols in Molecular Biology, 1994).

In some embodiments, antibodies may be screened against known related polypeptides to isolate an antibody population that specifically binds to BiP polypeptides, respectively. For example, antibodies specific to human BiP polypeptides will flow through a column composing BiP related proteins (with the exception of MICA) adhered to insoluble matix under appropriate buffer conditions. Such screening allows isolation of polyclonal and monoclonal antibodies non crossreactive to closely related polypeptides (Antibodies A Laboratory Manual, Harlow and Lane (eds), Cold Spring Harbor Laboratory Press, 1988, Current Protocols in Immunology, Cooligan et al (eds), National Institutes of Health, John Wiley and Sons, Inc, 1995). Screening and isolation of specific antibodies is well known in the art (see, Fundamental Immunology, Paul (eds), Raven Press, 1993, Getzoff et al, Adv m Immunol 43 1 98, 1988, Monoclonal Antibodies Principles and Practice, Godmg, J W (eds), Academic Press Ltd, 1996, Benjamin et al, Ann Rev Immunol 2 67-101, 1984). Representative examples of such assays include concurrent Immunoelectrophoresis, radioimmunoassay (RIA), radioimmunoprecrpitation, enzyme-lmked immunosorbent assay (ELISA), dot blot or Western blot assay, inhibition or competition assay, and sandwich assay.

Antibodies can be purified by chromatographic methods known to those of skill m the art, including ion exchange and gel filtration chromatography (for example, Came et al, Protein Expr Purif (1996) 8(2) 159 166). Alternatively or in addition, antibodies can be purchased from commercial sources, for example, Invitrogen (Carlsbad, Calif.), MP Biomedicals (Solon, Ohio), Nventa Biopharmaceuticals (San Diego, Calif.) (formerly Stressgen), Serologicals Corp (Norcross, Ga.).

The BiP -modulator can include a monoclonal antibody that recognizes a single epitope or can be any combination of monoclonal or polyclonal antibodies recognizing one of more different BiP epitopes. Thus the BiP -modulator can include antibodies that recognize 2, 3, 4, 5, 6, 7, 8, 10, 20 or more different BiP epitopes.

In some embodiments, antibodies may act as BiP antagonists. For example, in some embodiments the antibodies can disrupt the receptor/ligand interactions with BiP either partially or fully. In some embodiments, antibodies are provided that modulate ligand activity or receptor activity by at least 95%, at least 90%, at least 85%, at least 80%, at least 75%, at least 70%, at least 60%, or at least 50% compared to the activity in the absence of the antibody.

In some embodiments neutralizing antibodies are provided. In some embodiments the neutralizing antibodies act as receptor antagonists, i. e, inhibiting either all or a subset of the biological activities of the ligand-mediated receptor activation. In some embodiments the antibodies may be specified as agonists, antagonists or inverse agonists for biological activities comprising the specific biological activities of the peptides disclosed herein.

### Compositions and Methods for Eliciting an BiP Immune Response

The invention also provides compositions and methods for treating cancer in a subject by eliciting an immune response against BiF polypeptides. The terms “elicit,” “stimulate,” and “induce” are used interchangeably to denote the generation of a de novo immune response in a subject or to denote the enhancement of the strength or persistence of an existing immune response. The compositions of the invention contain, as an immunogenic component (also referred to herein as an “immunogen”), at least one BiP peptide.

In the context of the invention, an epitope is a portion of an antigenic molecule capable of eliciting an immune response to the molecule, preferably an antibody-secreting B cell mediated response, or which can be bound by an antibody. These antibodies enhance the activity of NK cells and CD8 T cells against cancer cells by inhibiting cleavage of MICA proteins from cancer cells.

The invention provides a vaccine composition suitable for administration to a human comprising, as an immunogenic component, at least one BiP peptide.

In one embodiment, the peptide epitopes are in the form of a structurally constrained loop. In one embodiment, the peptides retain their native secondary structure, for example in the form of one or more loops. In one embodiment, the loop is created using either a disulfide bond or a chemical linker. Preferably, the loop is adapted to mimic the three-dimensional conformation of the BiP epitope on the human protein.

In another embodiment, the vaccine composition comprises a nucleic acid encoding one or more of the BiP peptides. The nucleic acid may be in the form of an expression vector, for example a plasmid or a viral vector, or the nucleic acid may be packaged into nanoparticles. In one embodiment, the nucleic acid is delivered to a subject by injection. In one embodiment, the nucleic acid is injected as purified DNA or in form of nanoparticles. In one embodiment, modified immune cells which have been modified to express the nucleic acid are injected. In one embodiment, the immune cells are modified via transfection or infection in vitro with a vector comprising the nucleic acid.

In one embodiment, the vaccine composition comprises, as its immunogenic component, a plurality of BiP peptides. In one embodiment, the at least one peptide or the plurality of peptides is conjugated to a second peptide containing an MHC-II epitope. Preferably, the amino acid sequence of the second peptide consists of 25 amino acids or less, or 15 amino acids or less. In specific embodiments, the second peptide consists of 9-12 amino acids, 10-18 amino acids, or 8-18 amino acids. Preferably, the second peptide contains a T cell epitope or a B cell epitope. In one embodiment, the T cell epitope is a T helper cell epitope effective to enhance B cell differentiation into antibody-producing plasma cells or a cytotoxic T cell epitope. In one embodiment, the epitopes are overlapping epitopes for different MHC alleles or epitopes presented by many MHC allotypes. In another embodiment, the epitopes are peptides presented by different MHC alleles.

The peptides which form or are incorporated into the vaccine compositions of the invention are preferably purified from contaminating chemical precursors, if chemically synthesized, or substantially free of cellular material from the cell or tissue source from which they are derived. In a specific embodiment, the peptides are 60%, preferably 65%, 70%, 75%, 80%, 85%, 90%, 95%, or 99% free of contaminating chemical precursors, proteins, lipids or nucleic acids. In a preferred embodiment, the peptides are substantially free of contaminating virus. Preferably, each composition for administering to a subject is at least 95%, at least 97%, or at least 99% free of contaminating virus.

In one embodiment, the at least one peptide or the plurality of peptides of a vaccine composition of the invention comprises or consists of one or more peptides that is at least 90%, at least 95%, at least 98%, or at least 99% identical to a BiP peptide. In this context, the term “similar” refers to amino acid sequence similarity which is defined according to the number of conservative and non-conservative amino acid changes in a query sequence relative to a reference sequence. Conservative and non-conservative amino acid changes are known in the art. See, for example, W. R. Taylor, The Classification of Amino Acid Conservation, J. Theor. Biol. 1986 119:205-218, and D. Bordo and P. Argos, Suggestions for “Safe” Residue Substitutions in Site-Directed Mutagensis, 1991 J. Mol. Biol. 217:721-729. Generally, a conservative amino acid change refers to a substitution of one amino acid for another amino acid having substantially similar chemical properties, specifically with reference to the amino acid side chains. A non-conservative change refers to a substitution of one amino acid for another amino acid having substantially different chemical properties. Generally, conservative substitutions are those recognized in the art as being unlikely to affect the overall structure or biological function of the polypeptide, while non-conservative changes are recognized as more likely to affect structure and function.

Non-limiting examples of a conservative amino change include substitution of amino acids within the following groups: aliphatic, aromatic, polar, nonpolar, acidic, basic, phosphorylatable hydrophobic, hydrophilic, small nonpolar, small polar, large nonpolar, and large polar. Non-limiting examples of non-conservative amino acid changes include substitutions of amino acids between the foregoing groups.

In one embodiment, a conservative amino acid change is a substitution in which the substitution matrix for the pair of residues has a positive value. Examples of amino acid substitution matrices are known in the art, for example the BLOSUM50 matrix or the PAM250 matrix (see W. A. Pearson, Rapid and Sensitive Sequence Comparison with FASTP and FASTA, Meth. Enzymology, 1990 183:63-98, ed. R. Doolittle, Academic Press, San Diego). For further examples of scoring matrices and a comparison between them see M. S. Johnson and J. P. Overington, 1993, A Structural Basis for Sequence Comparisons: An Evaluation of Scoring Methodologies, J. Mol. Biol. 233:716-738.

In a preferred embodiment, a conservative amino acid change is a substitution of one amino acid for another amino acid within the same chemical group wherein the groups are selected from neutral and polar amino acids (Ser, Thr, Pro, Ala, Gly, Asn, Gln), negatively charged and polar amino acids (Asp, Glu), positively charged and polar amino acids (His, Arg, Lys), nonpolar amino acids lacking a ring structure (Met, Ile, Leu, Val), nonpolar amino acids having a ring structure (Phe, Tyr, Trp), and Cysteine.

In one embodiment, the vaccine composition comprises as its immunogenic component the chimeric protein displayed on the surface of a viral capsid, such as a Hepatitis B core capsid.

In one embodiment, the vaccine composition of the invention comprises as its immunogenic component a chimeric protein which consists of two or more BiP peptide epitopes selected placed into an immunoglobulin (Ig) domain having a similar overall immunoglobulin fold compared to MICA. In one embodiment, the Ig domain is an Ig domain selected from one of the following: UL18 (human CMV), the C-terminal Ig domain of IFN-alpha/beta binding protein C12R (poxvirus decoy receptor, PDB ID:30Q3), the N-terminal Ig domain of outer capsid protein from a T4-like bacteriophage (Hoc, PDB ID: 3SHS), and the human CMV protein US2 (PDB ID: 11M3).

In one embodiment consistent with any of the foregoing embodiments, the vaccine composition of the invention may comprise one or more polynucleotide sequences encoding the BiP. In a further embodiment, the DNA encoding the one or more BiP epitopes is in the form of a nanoparticle comprising the DNA.

In one embodiment, the vaccine composition comprises or is in the form of a protein scaffold and the at least one peptide or the plurality of peptides is contained within the scaffold. A particularly preferred scaffold is a porous, poly-lactide-co-glycolide (PLG) polymer scaffold. In one embodiment, the scaffold further comprises one or both of a GM-CSF protein and a Toll-like receptor agonist. In one embodiment, the Toll-like receptor agonist comprises or consists of unmethylated CpG oligonucleotides (a TLR9 agonist). The scaffold may also contain autologous tumor cell lysates, where autologous is with reference to the subject being treated (i.e., lysates of the subject's own tumor cells). In one embodiment, the scaffold is the WDVAX scaffold described in US 2013/0202707, WO 2011/063336, and US 2012/0100182. The scaffold is also described in Nature Materials, published online 11 Jan. 2009 DOI: 10.1038/NMAT2357 and in Science Translation Medicine, Sci Transl Med 1, 8ra19 (2009); DOI: 10.1126 /scitranslmed.3000359.

The vaccine compositions of the invention may further comprise one or more pharmaceutically acceptable additives or adjuvants. In one embodiment, the vaccine composition does not comprise an adjuvant. In one embodiment, the one or more adjuvants is selected from the group consisting of an oil-based adjuvant, a CpG DNA adjuvant, a mineral salt adjuvant, a mineral salt gel adjuvant, a particulate adjuvant, a micro particulate adjuvant, a mucosal adjuvant, and a cytokine.

Adjuvants may comprise any number of delivery systems, for example, mineral salts, surface active agents, synthetic micro particles, oil-in-water emulsions, immunostimulatory complexes, liposomes, virosomes, and virus-like particles. Adjuvants further comprises one or more potentiators of the immune response such as microbial derivatives (e.g., bacterial products, toxins such as cholera toxin and heat labile toxin from E. coli, lipids, lipoproteins, nucleic acids, peptidogylcans, carbohydrates, peptides), cells, cytokines, (e.g., dendritic cells, IL-12, and GM-CSF), hormones, and small molecules. Adjuvants contemplated include, but are not limited to, oil-based adjuvants (e.g., Freund's adjuvant), CpG oligonucleotides (see Klinman 2003 Expert Rev. Vaccines 2:305-15) aluminum salt adjuvants, calcium salt adjuvants, emulsions and surfactant-based formulations (e.g., MF59, ASO2, montanide, ISA-51, ISA-720, and QA21). For a review of improvements in vaccine adjuvants, see Pashine et al. 2005, Nature Med. 11(4):S63-S68.

In one embodiment, the adjuvant comprises or consists of one or more toll-like receptor (TLR) agonists. In one embodiment, the TLR agonist is a pathogen associated agonist selected from the group consisting of triacylated lipopeptides (gram positive bacteria), Peptidoglycan (gram positive bacteria), bacterial lipoprotein, lipoteichoic acid, LPS (Porphyromonas gingivalis, Leptospira interrogans), GPI-anchor proteins (Trypanosoma cruzi), neisserial porins, hemagglutinin (MV), phospholipomannan (Candida), LAM (Mycobacteria), ssRNA virus (WNV), dsRNA virus (RSV, MCMV), LPS (Gram-negative bacteria), F-protein (RSV), mannan (Candida), glycoinositolphospholipids (Trypanosoma), envelope proteins (RSV and MMTV), flagellin (Flagellated bacteria), phenol-soluble modulin (Staphylococcus epidermidis), diacylated lipopeptides (Mycoplasma), LTA (Streptococcus), zymosan (Saccharomyces), viral ssRNA (Influenza, VSV, HIV, HCV), ssRNA from RNA virus, dsDNA viruses (HSV, MCMV), hemozoin (Plasmodium), and unmethylated CpG DNA (bacteria and viruses).

In one embodiment, the TLR agonist is a synthetic ligand selected from the group consisting of Pam3Cys, CFA, MALP2, Pam2Cys, FSL-1, Hib-OMPC, Poly I:C; poly A:U, AGP, MPL A, RC-529, MDF2β, CFA, flagellin, MALP-2, Pam2Cys, FSL-1, Guanosine analogs, imidazoquinolines (e.g. Imiquimod, Aldara® R848, esiquimod®), loxoribine, imidazoquinolines, Loxoribine, ssPolyU, 3M-012, and CpG-oligonucleotides.

### Methods of Treating/Preventing Cancer

Provided herein are methods for treating and/or preventing cancer or symptoms of cancer in a subject comprising administering to the subject a therapeutically effective amount of a BiP-modulating composition. The BiP modulating composition can include one or more anti-BiP antibodies. Additionaly, the BiP vaccine compositions decribed herein are generally useful for generating immune responses and as prophylactic vaccines or immune response-stimulating therapeutics. As used herein, “prophylaxis” can mean complete prevention of the symptoms of a disease, a delay in onset of the symptoms of a disease, or a lessening in the seventy of subsequently developed disease symptoms As used herein, “therapy” can mean a complete abolishment of the symptoms of a disease or a decrease in the seventy of the symptoms of the disease In some embodiments the cancer is a cancer associated with overexpression of MIC. In some embodiments, the cancer is melanoma, lung, breast, kidney, ovarian, prostate, pancreatic, gastric, and colon carcinoma, lymphoma or leukemia. In some embodiments, the cancer is melanoma. In some embodiments, the cancer is a plasma cell malignancy, for example, multiple myeloma (MM) or pre-malignant condition of plasma cells. In some embodiments the subject has been diagnosed as having a cancer or as being predisposed to cancer.

The compositions disclosed herein are useful therapeutics for the treatment of pre-malignant disorders that carry with them a risk of progression to malignancy. Examples of such disorders include, without limitation, dysplasia, hyperplasia, and plasma cell disorders such as monoclonal gammopathy of undetermined significance (MGUS) and smoldering multiple myeloma (SMM).

Symptoms of cancer are well-known to those of skill m the art and include, without limitation, unusual mole features, a change in the appearance of a mole, including asymmetry, border, color and/or diameter, a newly pigmented skin area, an abnormal mole, darkened area under nail, breast lumps, nipple changes, breast cysts, breast pain, death, weight loss, weakness, excessive fatigue, difficulty eating, loss of appetite, chronic cough, worsening breathlessness, coughing up blood, blood in the urine, blood m stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, pam), pam, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreatic metastases, difficulty swallowing, and the like.

The methods disclosed herein can be applied to a wide range of species, e g, humans, non-human primates (e g, monkeys), horses, cattle, pigs, sheep, deer, elk, goats, dogs, cats, rabbits, guinea pigs, hamsters, rats, and mice.

The compositions can be administered directly to a mammal. Generally, the antibodies can be suspended in a pharmaceutically-acceptable earner (e g, physiological saline) A composition can be made by combining any of the BiP-modulating compositions provided herein with a pharmaceutically acceptable carriers. Such carriers can include, without limitation, sterile aqueous or non-aqueous solutions, suspensions, and emulsions Examples of non-aqueous solvents include mineral oil, propylene glycol, polyethylene glycol, vegetable oils, and injectable organic esters, for example. Aqueous earners include, without limitation, water, alcohol, saline, and buffered solutions Preservatives, flavorings, and other additives such as, for example, antimicrobials, anti-oxidants, chelating agents, inert gases, and the like also may be present. It will be appreciated that any material described herein that is to be administered to a mammal can contain one or more pharmaceutically acceptable carriers.

Any composition described herein can be administered to any part of the host's body. A composition can be delivered to, without limitation, the joints, nasal mucosa, blood, lungs, intestines, muscle tissues, skin, or peritoneal cavity of a mammal. In addition, a composition can be administered by intravenous, intraperitoneal, intramuscular, subcutaneous, intramuscular, intrarectal, intravaginal, intrathecal, intratracheal, intradermal, or transdermal injection, by oral or nasal administration, by inhalation, or by gradual perfusion over time. In a further example, an aerosol preparation of a composition can be given to a host by inhalation.

The dosage required depends on the route of administration, the nature of the formulation, the nature of the patient's illness, the subject's size, weight, surface area, age, and sex, other drugs being administered, and the judgment of the attending physician Suitable dosages are m the range of 0 01-1,000 μg/kg. Wide variations in the needed dosage are to be expected in view of the variety of BiP-modulating compositions available and the differing efficiencies of various routes of administration. Variations in these dosage levels can be adjusted using standard empirical routines for optimization as is well understood in the art. Administrations can be single or multiple (e g, 2- or 3-, 4-, 6-, 8-, 10-, 20-, 50-, 100, 150-, or more fold). Encapsulation of the composition in a suitable delivery vehicle [e g, polymeric microparticles or implantable devices) may increase the efficiency of delivery.

The duration of treatment with any composition provided herein can be any length of time from as short as one day to as long as the life span of the host (e g, many years). For example, BiP-modulating compositions can be administered once a month for three months or once a year for a period often years. It is also noted that the frequency of treatment can be variable. For example, BiP-modulating compositions can be administered once (or twice, three times, etc) daily, weekly, monthly, or yearly BiP modulating compositions can be administered together, i e, at the same point in time or sequentially.

An effective amount of any composition provided herein can be administered to a host. The term “effective” as used herein refers to any amount that induces a desired immune response while not inducing significant toxicity in the host. Such an amount can be determined by assessing a host's immune response after administration of a known amount of a particular composition In addition, the level of toxicity, if any, can be determined by assessing a host's clinical symptoms before and after administering a known amount of a particular composition. It is noted that the effective amount of a particular composition administered to a host can be adjusted according to a desired outcome as well as the host's response and level of toxicity. Significant toxicity can vary for each particular host and depends on multiple factors including, without limitation, the host's disease state, age, and tolerance to pain.

Antibodies can also be administered to a subject via in vivo therapeutic antibody gene transfer as discussed by Fang et al (2005), Nat Biotechnol 23, 584-590 For example recombinant vectors can be generated to deliver a multicistronic expression cassette comprising a peptide that mediates enzyme independent, cotranslational self cleavage of polypeptides placed between MAb heavy and light chain encoding sequences. Expression leads to stochiometicc amounts of both MAb chains.

In addition, clinical methods that can assess the degree of a particular disease state can be used to determine if a desired immune response is induced. For example, in a cancer patient, a reduction in tumor burden or a delay in the recurrence or metastasis can indicate a desired immune response in a patient treated with a BiP-modulating composition.

Also provided are methods of inhibiting cancer in a patient The methods comprise determining if the patient is a candidate for BiP therapy as described herein and administering a therapeutically effective amount of one or more BiP modulators to the patient if the patient is a candidate for BiP therapy. Further provided are methods of inhibiting cancer m a patient diagnosed or suspected of having a cancer The methods comprise administering a therapeutically effective amount of one or more BiP modulators to the patient. Also provide are methods of modulating one or more symptoms of cancer in a patient comprising administering to said patient a therapeutically effective amount of one or more MICA modulators.

Methods to prophylactically treat a patient who is predisposed to develop cancer, a cancer metastasis or who has had a metastasis and is therefore susceptible to a relapse or recurrence are disclosed The methods are particularly useful in high-risk individuals who, for example, have a family history of cancer or of metastasizing tumors, or show a genetic predisposition for a cancer metastasis In some embodiments the tumors are MICA-related tumors. Additionally, the methods are useful to prevent patients from having recurrences of MICA related tumors who have had MICA-related tumors removed by surgical resection or treated with a conventional cancer treatment. Also provided are methods of inhibiting cancer progression and/or causing cancer regression comprising administering to the patient a therapeutically effective amount of an BiP modulator.

In some embodiments, the patient in need of anti-cancer treatment can be treated with the BiP modulators described herein in conjunction with one or more antibodies directed at targets other than BiP. Suitable targets can include cancer cell surface molecules, e g, the MICA, EGF receptor, VEGF, HER-2, CD20, c-Met, ErbB3, angiopoietins, and ganglosides such as GM2. In some embodiments, the patient in need of anti-cancer treatment is treated with the BiP modulators described herein in conjunction with chemotherapy and/or radiation therapy. For example, following administration of the BiP modulators, the patient may also be treated with a therapeutically effective amount of anti-cancer radiation. In some embodiments chemotherapeutic treatment is provided in combination with BiP modulator. In some embodiments BiP modulators are administered in combination with chemotherapy and radiation therapy

Methods of treatment comprise administering single or multiple doses of one or more BiP modulators to the patient. In some embodiments the BiP modulators are administered as injectable pharmaceutical compositions that are sterile, pyrogen free and comprise the BiP modulators in combination with a pharmaceutically acceptable carrier or diluent.

In some embodiments, the therapeutic regimens described herein are used with conventional treatment regimens for cancer including, without limitation, surgery, radiation therapy, hormone ablation and/or chemotherapy. Administration of the BiP modulators described herein may take place pρor to, simultaneously with, or after conventional cancer treatment In some embodiments, two or more different BiP modulators are administered to the patient.

Also provided are methods of monitoring the progression of pre-malignant disorders that have the potential for progression to malignancy, for example, plasma cell disorders such as monoclonal gammopathy of undetermined significance (MGUS) and smoldeing multiple myeloma (SMM). More specifically, a patient having a pre malignant plasma cell disorder can be identified as being at risk for progression of the pre-malignant plasma cell disorder to a malignancy by assessing the levels of MICA or anti MICA antibodies in the individual MICA can be either cell-associated MICA, i.e., intracellular or cell surface MICA, or sMICA. In some embodiments, an individual who does not express or who expresses low levels of cell-associated MICA or anti MICA antibodies relative to a reference sample can be classified as being at risk for progression to malignancy. In some embodiments, an individual who expresses elevated levels of sMICA relative to a reference sample can be classified as being at risk for progression to malignancy.

The level of MICA or anti-MICA antibodies can be measured in any biological sample known m the art to contain MICA or anti-MICA antibodies. Examples of biological samples include, without limitation, whole blood, serum, blood plasma, peripheral blood mononuclear cells (PBMCs) and bone marrow aspirates. Biological samples can be collected from an individual using any standard method known in the art that results m the preservation of MICA or anti-MICA antibodies. Blood samples can be obtained via venous puncture techniques. Serum samples can be prepared from whole blood using standard methods such as centrifuging blood samples that have been allowed to clot Plasma samples can be obtained by centifuging blood samples that were treated with an anti-coagulant such as heparin PBMCs and bone marrow aspirates can be processed by Ficoll-Hypaque density gradient centrifugation. Biological samples can be assayed for MICA or anti-MICA antibodies immediately following collection.

Alternatively, or in addition, a biological fluid sample can be stored for later analysis using methods known in the art that preserve MICA or anti-MICA antibodies, e g, freezing, drying, freeze drying.

After determining the levels of MICA or anti-MICA antibodies m a biological sample, these levels can be compared with those of a control sample. A control sample can be a one or more samples taken from the same individual at anearlier point m time. Alternatively or in addition a control sample can be a standard reference level Standard reference levels typically represent the average MICA or anti-MICA antibody levels derived from a large population of individuals. The reference population may include individuals of similar age, body size, ethnic background or general health as the individual in question.

In general, an elevated level of MICA or anti-MICA antibodies can be any level of MICA or anti-MICA antibodies that is greater than either the level of MICA or anti-MICA antibodies found m a control sample or the average level of MICA or anti-MICA antibodies found in samples from a population of normal healthy individuals. A reduced level of MICA or anti-MICA antibodies can be any level of MICA or anti-MICA antibodies antigen that is less than either the level of MIC A or anti-MICA antibodies found in a control sample or the average level of MICA or anti-MICA antibodies found in samples from a population of normal healthy individuals. Any population size can be used to determine the average level of MICA or anti-MICA antibodies found m samples from a population of normal healthy individuals. For example, a population of 2, 3, 4, 5, 10, 15, 20, 25, 30, 40, 50, 100, 150, 200, 250 or more individuals can be used to determine the average level of MICA or anti-MICA antibodies in samples from a population of normal healthy individuals.

An elevated level of MICA or anti-MICA antibodies can be 1, 2, 3, 4, 5, 10, 20, or more percent higher than that level found in a control sample or the average level of MICA or anti-MICA antibodies found in samples from a population of normal healthy individuals. In some cases, an elevated level of MICA or anti-MICA antibodies can be 1, 2, 3, 4, 5, 10, or more fold higher than that level found in a control sample or the average level of MICA or anti-MICA antibodies found in samples from a population of normal healthy mammals. A reduced level of MICA or anti-MICA antibodies can be 10, 20, 30, 50, 60, 70, 80, 90, 100, 150 or more percent lower than that level found in a control sample or the average level of MICA or anti-MICA antibodies found m samples from a population of normal healthy mammals. In some cases, a reduced level of MICA or anti-MICA antibodies can be 1, 2, 3, 4, 5, 10, 20, 50 or more fold lower than that level found in a control sample or the average level of MICA or anti-MICA antibodies found in samples from a population of normal healthy mammals. In some cases, a reference chart can be used to determine whether or not a particular level of MICA or anti MICA antibodies in a sample is reduced, normal, or elevated relative to a control sample or a larger population. For example, a reference chart can contain the normal range of MICA or anti MICA antibodies found m healthy individuals of the same age, gestational age, ethnic background or general health as the individual in question Using this reference chart, any level of MICA or anti-MIC A antibodies measured in a sample can be classified as being an reduced, normal, or elevated relative to a control sample or a larger population.

Alternatively, or in addition, the level of MICA or anti-MICA antibodies in a biological sample can be “normalized” against one another or against the level of one or more additional biological markers. The values for the level of cell-associated MICA, sMICA or anti-MICA antibodies may be expressed as a ratio and the ratios may be compared to similar ratio obtained for a reference sample or population That is, the levels of the additional marker can be evaluated in parallel with those of MICA or anti-MICA antibodies, either at the same time or on a separate occasion. The additional marker can serve as an internal control for sample preparation, handling and storage as well as day-to-day assay variability.

Once the relative level of MICA or anti MICA antibodies in an individual relative to that of a reference sample has been calculated, the individual's relative risk for progression to malignancy can be assessed. Any statistical method known in the art for evaluating relative risk may be used, for example receiver operator characteristic curve analysis. The receiver operated characteristics (ROC) value describes the balance between the sensitivity (i.e., the number of hits detected) and the specificity (i.e., the accuracy) of a test. These two variables may also be considered positive predictive value and negative predictive value, and are correlated with diagnostic accuracy. The ROC curve shows the relationship of the probability of a positive test, given no disease, to the probability of a positive test, given disease An ROC cutoff value is chosen to maximize diagnostic accuracy of the test in question. Following assessment of relative risk for progression, appropriate therapies, such as the administration of anti-BiF antibodies described above, as well as conventional cancer therapies can be initiated.

### Combination Therapy

In some embodiments compositions comprising two or more BiP modulators are provided. In some embodiments the BiP modulators are monoclonal antibodies. Compositions comprising two or more anti-BiP antibodies may be administered to persons or mammals suffering from, or predisposed to suffer from, cancer. In other embodiments, a BiP modulator composition of the invention is administered as part of a therapeutic regimen that includes surgery, a chemotherapeutic agent, or radiation therapy, an immunotherapy, or any combination of the foregoing.

Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

The present invention also provides methods for the treatment or prophylaxis of cancer which comprise administering a vaccine composition of the invention to a subject in need thereof, along with one or more additional therapeutic agents or therapeutic regimens. In one embodiment, a vaccine composition of the invention is administered as part of a therapeutic regimen that includes surgery, a chemotherapeutic agent, or radiation therapy, an immunotherapy, or any combination of the foregoing.

In one embodiment, the therapeutic regimen comprises or further comprises a one or more immunostimulatory agents. In one embodiment, the one or more immunostimulatory agents is selected from the group consisting of an anti-CTLA-4 antibody or peptide, an anti-PD-1 antibody or peptide, an anti-PDL-1 antibody or peptide, an anti-OX40 (also known as CD134, TNFRSF4, ACT35 and/or TXGP1L) antibody or peptide, an anti-GITR (also known as TNFRSF18, AITR, and/or CD357) antibody or peptide, an anti-LAG-3 antibody or peptide, and/or an anti-TIM-3 antibody or peptide.

In one embodiment, the one or more immunostimulatory agents is selected from an anti-MICA antibody described in WO 2013/049517 or WO 2008/036981. In one embodiment, the one or more immunostimulatory agents is selected from CM33322 Ab4, CM33322 Ab28, and CM33322 Ab29, which are described in U.S. Provisional Application Nos. 61/792,034 and 61/913,198 and in U.S. application Ser. No. 14/025,573.

In one embodiment, the therapeutic regimen comprises or further comprises one or more cytokines. In one embodiment, the BiP modulator compositions or vaccine compositions of the invention comprise one or more cytokines. In one embodiment, at least one cytokine is an interleukin or an interferon. In one embodiment, at least one cytokine is an interleukin selected from the group consisting of IL-1.alpha., IL-1.beta., IL-2, IL-3, IL-4, IL-5, IL-6, IL-7, IL-8, IL-9, IL-11, IL-12, IL-13, IL-15, and IL-18. In another embodiment, at least one cytokine is an interferon selected from IFN.alpha., IFN.beta., and IFN.gamma.

In one embodiment, the BiP modulator composition or vaccine composition of the invention is administered as part of a therapeutic regimen that includes administering to the subject at least one chemotherapeutic agent selected from the group consisting of histone deacetylase inhibitors (“HDAC”) inhibitors, proteasome inhibitors, alkylating agents, and topoisomerase inhibitors.

In one embodiment, the chemotherapeutic agent is an HDAC inhibitor selected from the group consisting of hydroxamic acid, Vorinostat (Zolinza), suberoylanilide hydroxamic acid (SAHA)(Merck), Trichostatin A (TSA), LAQ824 (Novartis), Panobinostat (LBH589) (Novartis), Belinostat (PXD101)(CuraGen), ITF2357 Italfarmaco SpA (Cinisello), Cyclic tetrapeptide, Depsipeptide (romidepsin, FK228) (Gloucester Pharmaceuticals), Benzamide, Entinostat (SNDX-275/MS-275)(Syndax Pharmaceuticals), MGCD0103 (Celgene), Short-chain aliphatic acids, Valproic acid, Phenyl butyrate, AN-9, pivanex (Titan Pharmaceutical), CHR-3996 (Chroma Therapeutics), and CHR-2845 (Chroma Therapeutics).

In one embodiment, the chemotherapeutic agent is a proteasome inhibitor selected from the group consisting of Bortezomib, (Millennium Pharmaceuticals), NPI-0052 (Nereus Pharmaceuticals), Carfilzomib (PR-171)(Onyx Pharmaceuticals), CEP 18770, and MLN9708.

In one embodiment, the chemotherapeutic agent is an alkylating agent such as mephalan.

In one embodiment, the chemotherapeutic agent is a topoisomerase inhibitor such as Adriamycin (doxorubicin).

In one embodiment, the therapeutic regimen comprises or further comprises one or more of chemotherapy, radiation therapy, cytokines, chemokines and other biologic signaling molecules, tumor specific vaccines, cellular cancer vaccines (e.g., GM-CSF transduced cancer cells), tumor specific monoclonal antibodies, autologous and allogeneic stem cell rescue (e.g., to augment graft versus tumor effects), other therapeutic antibodies, molecular targeted therapies, anti-angiogenic therapy, infectious agents with therapeutic intent (such as tumor localizing bacteria) and gene therapy.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating or alleviating a symptom of cancer by administering to a subject an effective amount of a BiP modulating composition.

2. The method of claim 1, wherein the BiP modulating composition is a BiP antibody.

3. The method of claim 2, further comprising administering an antibody specific for a charperone protein expressed on the surface of a tumor cell.

4. The method claim 1, wherein the BiP modulator composition reduces the level of soluble MIC in the subject.

5. The method of claim 3, wherein the reduction of the level of the soluble MICA or MICB

6. The method of claim 3, wherein the level of soluble MICA or MICB is the level in the serum.

7. A method of treating or alleviating a symptom by administering to a subject a BiP peptide in an amount sufficient to induce an anti-BiP immune response.

8. The method of claim 6, wherein the BiP peptide is conjugated to a carrier protein.

9. The method of claim 7, wherein the carrier protein is tetnus toxin or diphtheria toxin.

10. A method of treating or alleviating a symptom by administering to a subject a BiP protein or fragment thereof.

11. The method of claim 9, wherein the subject has been administered a MIC vaccine.

12. The method of claim 9, further comprising administering to said subject a MIC antibody.

13. The method of claim 11, wherein the MIC antibody is CM24002.

14. A method of treating or alleviating a symptom of cancer by administering to a subject an effective amount of a bispecific antibody that specifically binds to MIC and BiP.

15. A method of producing an immunogen comprising contacting a tumor specific antigen with a BiP polypeptide in an amount sufficient to induce partial unfolding of the antigen.

16. An immunogen comprising a cell co-expressing BiP and an antigen.

17. The immunogen of claim 16, wherein the antigen is an NKG2D ligand.

18. The immunogen of claim 17, wherein the NKG2D ligand is MIC.
